(19) United States
(12) Patent Application Publication
(10) Pub. No.: US 2023/0154628 A1
(43) Pub. Date: May 18, 2023

(54) ANALYSIS APPARATUS, ANALYSIS METHOD AND PROGRAM

(71) Applicant: NIPPON TELEGRAPH AND TELEPHONE CORPORATION, Tokyo (JP)

(72) Inventors: Tomu TOMINAGA, Tokyo (JP); Takeshi KURASHIMA, Tokyo (JP); Hiroyuki TODA, Tokyo (JP)

(73) Assignee: NIPPON TELEGRAPH AND TELEPHONE CORPORATION, Tokyo (JP)

(21) Appl. No.: 17/916,433

(22) PCT Filed: Apr. 7, 2020
(86) PCT No.: PCT/JP2020/015680

§ 371 Date: Sep. 30, 2022

(51) Int. Cl. G16H 50/80; G16H 50/70; G06N 5/022

(52) U.S. Cl. CPC G16H 50/80; G16H 50/70; G06N 5/022

(57) ABSTRACT

An analysis apparatus for analyzing a causal relationship between an incidence of a predetermined disease and a predetermined intervention includes a memory; and a processor configured to execute: converting a plurality of first parameters indicative of attributes of users belonging to a population, at least two of the parameters having correlations with a predetermined strength, to a plurality of second parameters without the correlations with the predetermined strength with each other; computing a predetermined score for each of the users, using the plurality of second parameters and a parameter indicative of presence or absence of the intervention; and clustering the users belonging to the population using the score, to analyze the causal relationship.

### TECHNICAL FIELD

The present invention relates to an analysis apparatus, analysis method, and a program.

### BACKGROUND ART

Propensity score analysis (or sometimes also called “propensity score analytics”), which is a type of statistical causal inference, has been known heretofore (see, for example, NPL 1). Propensity score analysis estimates a probability that a test subject has a specific factor from a plurality of covariates. This probability is called a propensity score. The propensity score is basically free of limitation by the number of covariates because of the nature of one-dimensional aggregation of covariates. Hence, propensity score analysis has an advantage that with a greater number of covariates, the causal inference can be performed more robustly.

### CITATION LIST

### Non Patent Literature

[NPL 1] Takahiro Hoshino and Kazuo Shigemasu, “Estimation of Causal Effect and Adjustment of Survey Data using Propensity Scores,” The Japanese Journal of Behaviormetrics, Volume 31 Issue 1, 2004, pp. 43-61

### SUMMARY OF THE INVENTION

### Technical Problem

When estimating a propensity score from covariates, however, sometimes (strong) correlations are confirmed among the covariates. In such a case, it is necessary to exclude one of the covariates that are correlated to each other from the analysis. In particular, the larger the number of covariates to be used for the analysis, the higher the possibility of multicollinearity. Therefore, when performing propensity score analysis, while as many covariates as possible should be obtained, it is necessary to prevent occurrence of multicollinearity without excluding any of the covariates.

One embodiment of the present invention was made in view of the issue described above, with an object to prevent occurrence of multicollinearity.

### Means For Solving the Problem

To achieve the above object, an analysis apparatus according to one embodiment is an analysis apparatus for analyzing a causal relationship between an incidence of a predetermined disease and a predetermined intervention, characterized by having: a conversion unit configured to convert a plurality of first parameters indicative of attributes of users belonging to a population, at least two of the parameters having a correlation with a predetermined strength, to a plurality of second parameters without the correlation with the predetermined strength with each other; a computing unit configured to compute a predetermined score for each of the users, using the plurality of second parameters and a parameter indicative of presence or absence of the intervention; and a clustering unit configured to cluster the users belonging to the population using the score, to analyze the causal relationship.

### Effects of the Invention

Occurrence of multicollinearity can be prevented.

### BRIEF DESCRIPTION OF DRAWINGS

FIG. 1 is a diagram illustrating one example of a functional configuration of an analysis apparatus according to the embodiment.

FIG. 2 is a diagram illustrating one example of sample data stored in a sample DB.

FIG. 3 is a flowchart illustrating one example of a flow of analysis process according to the embodiment.

FIG. 4 is a diagram illustrating an example of a hardware configuration of the analysis apparatus according to the embodiment.

### DESCRIPTION OF EMBODIMENTS

Hereinafter, one embodiment of the present invention will be described. This embodiment will describe an analysis apparatus 10 that converts covariates to mutually uncorrelated variables while retaining a relationship between the covariates when performing propensity score analysis so that occurrence of multicollinearity can be prevented.

Note that this embodiment will describe a case, as one example, where a causal effect of smoking experience on the development of lung cancer is validated by propensity score analysis using sample data acquired from an observational study. This is merely one example, and the analysis apparatus 10 according to this embodiment can be similarly applied to other cases where a causal effect between a given intervention (factor) and a given outcome is validated by propensity score analysis.

<Functional Configuration>

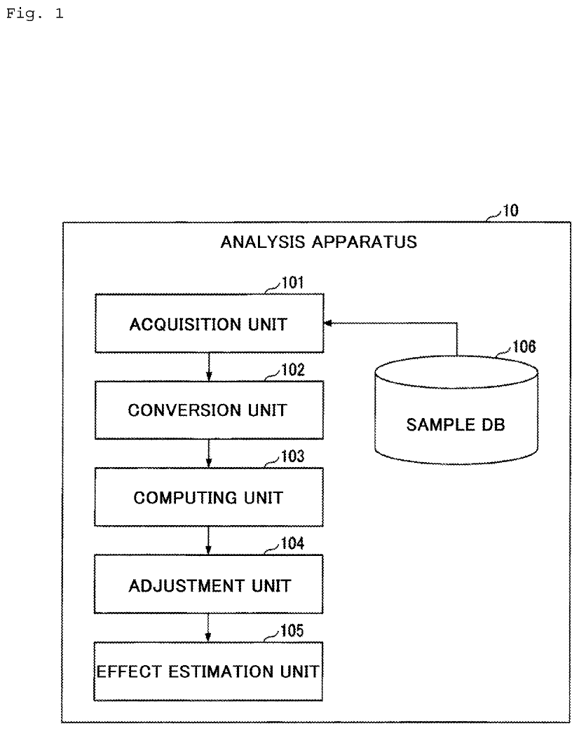

First, a functional configuration of the analysis apparatus 10 according to the embodiment will be described with reference to FIG. 1. FIG. 1 is a diagram illustrating one example of the functional configuration of the analysis apparatus 10 according to the embodiment.

As illustrated in FIG. 1, the analysis apparatus 10 according to this embodiment includes an acquisition unit 101, a conversion unit 102, a computing unit 103, an adjustment unit 104, an effect estimation unit 105, and a sample DB 106.

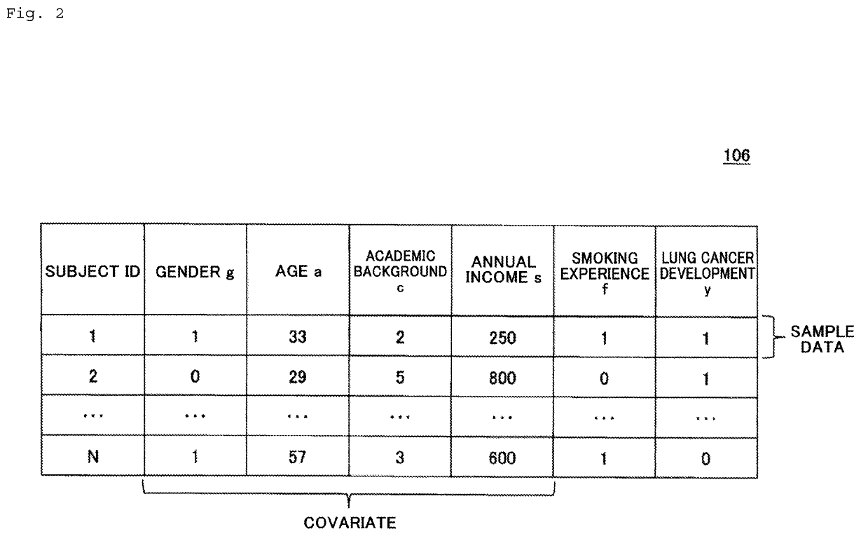

The sample DB 106 stores multiple sets of sample data (i.e., population of sample data) used for the propensity score analysis. Now, one example of sample data stored in the sample DB 106 will be described with reference to FIG. 2. FIG. 2 is a diagram illustrating one example of sample data stored in the sample DB 106.

As illustrated in FIG. 2, the sample DB 106 stores multiple sets of sample data, each sample data set containing a plurality of items. For example, in the case illustrated in FIG. 2, each sample data set contains items “subject ID,” “gender g,” “age a,” “academic background c,” “annual income s,” “smoking experience f,” and “lung cancer development y.” The items can also be called parameters.

In this embodiment, among items included in sample data, the items “subject ID,” “gender g,” “age a,” “academic background c,” and “annual income s” are covariates, while the “smoking experience f” is a treatment variable and the “lung cancer development y” is an outcome variable. Meanwhile, the subject ID is identification information that uniquely identifies a subject (sample or user). In this embodiment, the subject ID is represented as i (i=1, . . . , N). Treatment variables are variables that indicate presence or absence of an intervention (factor) by their values for allocating sample data to either a treated group or a control group (the treated group and the control group may also be referred to as the exposed group and the unexposed group, respectively, for example). In general, parameters expected to have a causal relationship with an outcome variable are set as treatment variables.

Note that values 0 and 1 under “gender g” indicate male and female, respectively, for example, values under “age a” indicate ages, values under “academic background c” indicate final academic records, and values under “annual income s” indicate annual salaries. Values 0 and 1 under “smoking experience f” respectively indicate absence and presence of smoking experience, for example. Values 0 and 1 respectively indicate “absence and presence of development of lung cancer y.”

Hereinafter, sample data of a subject ID “i” will be expressed as sample data i, and the gender g, age a, academic background c, annual income s, smoking experience f, and lung cancer development y contained in the sample data i will be expressed as gᵢ, aᵢ, cᵢ, sᵢ, fᵢ, and yᵢ, respectively. A vector having covariates as its elements will be referred to as covariate vector. A covariate vector having the covariates gᵢ, aᵢ, cᵢ, and sᵢ contained in the sample data i will be expressed as xᵢ=(gᵢ, aᵢ, cᵢ, sᵢ).

As described above, the sample DB 106 stores a plurality of sample data sets, each containing at least two or more covariates (parameters). Note, “gender g,” “age a,” “academic background c,” and “annual income s” are merely examples of covariates, and various other parameters obtained by an observational study (e.g., parameters indicative of a variety of subjects' attributes such as family configuration, birthplace, nationality, hobby, occupation, average sleep time, whether they drink or not, etc.) can be set as covariates.

The acquisition unit 101 acquires N set(s) of sample data that is to be the object of propensity score analysis from the sample DB 106.

The conversion unit 102 converts each of the covariates contained in each sample data i acquired by the acquisition unit 101 to mutually uncorrelated variables (parameters) while retaining relationships among the covariates. In other words, the conversion unit 102 converts each covariate vector xᵢ to a vector x′ᵢ having mutually uncorrelated variables as its elements while retaining relationships among the covariates. Hereinafter, this converted vector x′ᵢ will be referred to as covariate principal component vector x′ᵢ.

The conversion unit 102 performs principal component analysis using covariate vectors x₁, . . . , x_{N}, for example, and for each covariate vector xᵢ, converts each of the elements gᵢ, aᵢ, cᵢ, and sᵢ of to a first principal component point Pcᵢ₁, a second principal component point Pcᵢ₂, a third principal component point Pcᵢ₃, and a fourth principal component point Pcᵢ₄, respectively. The covariate vector xᵢ=(gᵢ, aᵢ, cᵢ, sᵢ) is thus converted to a covariate principal component vector x′ᵢ=(Pcᵢ₁, Pcᵢ₂, Pcᵢ₃, Pcᵢ₄).

Note that in general, when the number of elements (i.e., number of covariates) of the covariate vector xᵢ is J, the covariate vector xᵢ may be converted to covariate principal component vector x′ᵢ by converting j-th element of the covariate vector xᵢ (where j=1, . . . , J) to a j-th principal component point Pcᵢⱼ.

The computing unit 103 estimates a propensity score by using the covariate principal component vectors x′ᵢ obtained by converting the covariate vectors xᵢ by means of the conversion unit 102. Specifically, the computing unit 103 computes (estimates) propensity scores eᵢ of sample data i by eᵢ=Pr(fᵢ=1|x′ᵢ). The propensity scores eᵢ can be computed using a known model (e.g., logistic regression, machine learning models such as random forest, Generalized Boosting Modeling, NN (Neural Network), etc.).

In this way, even when there are correlations among (certain) covariates, propensity scores can be computed (estimated) while avoiding multicollinearity by using covariate principal component vectors. In this embodiment, for example, even when the academic background c and annual income s have a high correlation coefficient (i.e., there is a strong correlation), propensity scores eᵢ can be computed (estimated) while avoiding multicollinearity by using the covariate principal component vectors x′ᵢ.

The adjustment unit 104 adjusts the covariates of a treated group and a control group by known techniques (e.g., matching, stratification, and the like) using the propensity scores eᵢ computed (estimated) by the computing unit 103, and reconstructs the treated group and control group. Namely, the adjustment unit 104 reconstructs the treated group and control group by grouping the sample data sets in each of the treated group and control group. In this way, a treated group and a control group having covariates (averages and the like) similar to each other are obtained. Grouping may also be referred to as clustering or classification.

In the case of using nearest neighbor matching, for example, a sample data set in a treated group (e.g., a set of sample data i where fᵢ=1) may be paired with a sample data set in a control group (e.g., a set of sample data i where fᵢ=0) having a closest propensity score, and the treated group and control group may be reconstructed by such pairing. In doing so, for example, a caliper (tolerance range) may be set to each of sample data belonging to the treated group before reconstruction, and sample data sets having propensity scores with a difference within the caliper may be matched up as pairs. These matching techniques are merely examples and any other matching techniques can be used.

Also, in the case of using stratification, for example, the treated group and control group may be reconstructed by dividing each of the treated group and control group into a plurality of subclasses based on the propensity scores. The number of subclasses may be any number. The number of subclasses is often set to 5, for example.

The effect estimation unit 105 estimates a causal effect by a known method (e.g., statistical test or the like), using the treated group and control group reconstructed by the adjustment unit 104. Thus a causal effect from an intervention (factor) to an outcome (in this embodiment, a causal effect between smoking experience f and development of lung cancer y) is estimated. Accordingly, for example, in this embodiment, it becomes possible to verify whether or not there is a causal relationship between smoking experience and incidence of lung cancer. As described above, in general, the propensity score analysis is often used for verification of whether or not there is actually a causal relationship between an intervention (factor) expected to have a causal relationship with a disease and the incidence of this disease.

<Analysis Process>

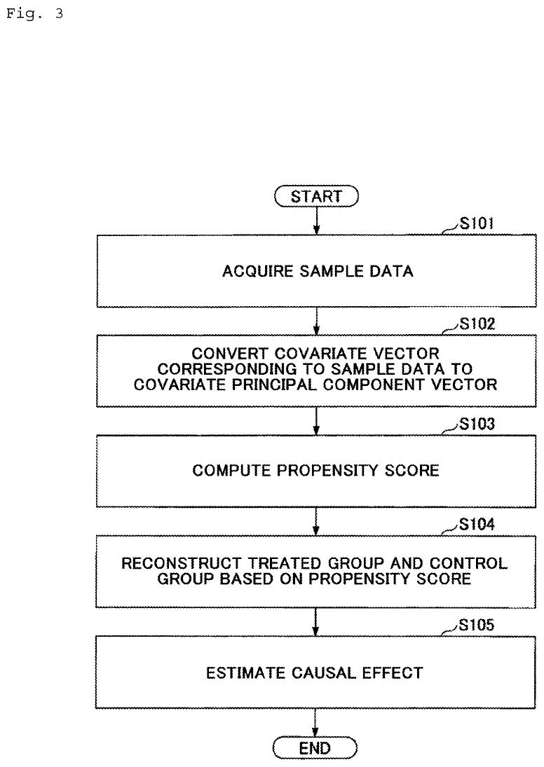

Next, a process flow when propensity score analysis is performed by the analysis apparatus 10 according to this embodiment will be described with reference to FIG. 3. FIG. 3 is a flowchart illustrating one example of a flow of analysis process according to the embodiment.

First, the acquisition unit 101 acquires N set(s) of sample data that is to be the object of propensity score analysis from the sample DB 106 (step S101).

Next, the conversion unit 102 converts covariate vectors xᵢ corresponding to the sample data i (where i=1, . . . , N) acquired at the above step S101 to covariate principal component vectors x′ᵢ (step S102).

Next, the computing unit 103 computes propensity scores eᵢ from the covariate principal component vectors x′ᵢ obtained at the above step 5102 (step S103).

Next, the adjustment unit 104 adjusts the covariates of the treated group and control group by a known technique using the propensity scores eᵢ computed at the above step S103, and reconstructs the treated group and control group (step S104).

Then, the effect estimation unit 105 estimates a causal effect by a known technique (step S105) using the treated group and control group obtained at the above step S104.

The analysis apparatus 10 according to this embodiment can thus estimate a propensity score while preventing occurrence of multicollinearity even when there are included covariates that are correlated to each other. Moreover, since the analysis apparatus 10 according to this embodiment converts covariate vectors to covariate principal component vectors, the covariates can be uncorrelated from each other without excluding covariates (i.e., without reducing the estimation accuracy of causal effect) and while keeping the relationship between the covariates.

Note that covariates having a strong correlation with each other raise the likelihood of occurrence of multicollinearity. In such a case, the use of the analysis apparatus 10 according to this embodiment is particularly effective. There is still a possibility of multicollinearity when the covariates having a weak correlation are included. Therefore, the use of the analysis apparatus 10 according to this embodiment can ensure that occurrence of multicollinearity is prevented irrespective of the degree of correlation.

<Hardware Configuration>

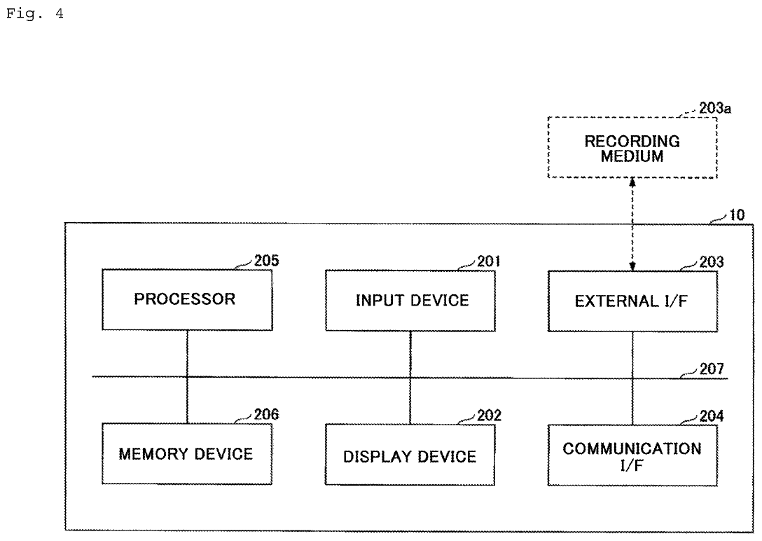

Lastly, a hardware configuration of the analysis apparatus 10 according to this embodiment will be described with reference to FIG. 4. FIG. 4 is a diagram illustrating an example of the hardware configuration of the analysis apparatus 10 according to the embodiment.

As illustrated in FIG. 4, the analysis apparatus 10 according to this embodiment is implemented as a common computer or a computer system and includes an input device 201, a display device 202, an external I/F 203, a communication I/F 204, a processor 205, and a memory device 206. These hardware components are connected to one another via a bus 207 so as to be able to communicate with each other.

The input device 201 is a keyboard, mouse, touchscreen and the like, for example. The display device 202 is a display and the like, for example. The analysis apparatus 10 can do without at least one of the input device 201 and the display device 202.

The external I/F 203 is an interface with an external device. The external device includes a recording medium 203a and the like. The analysis apparatus 10 can read or write data from or to the recording medium 203a and the like via the external I/F 203. The recording medium 203a may store one or more programs that implement(s) the functional units of the analysis apparatus 10 (acquisition unit 101, conversion unit 102, computing unit 103, adjustment unit 104, and effect estimation unit 105).

The recording medium 203a includes, for example, a CD (Compact Disc), DVD (Digital Versatile Disk), SD memory card (Secure Digital memory card), USB (Universal Serial Bus) memory card, and so on.

The communication I/F 204 is an interface for connecting the analysis apparatus 10 to a communication network. One or more programs that implement(s) the functional units of the analysis apparatus 10 may be obtained (downloaded) from a predetermined server device or the like via the communication I/F 204.

The processor 205 is one of various computing devices such as a CPU (Central Processing Unit) or GPU, for example. The functional units of the analysis apparatus 10 are implemented by one or more programs stored in the memory device 206 causing the processor 205 to perform the processing, for example.

The memory device 206 is one of various storage devices such as the HDD (Hard Disk Drive), SSD (Solid State Drive), RAM (Random Access Memory), ROM (Read Only Memory), flash memory, and so on, for example. The sample DB 106 of the analysis apparatus 10 can be implemented using the memory device 206, for example. The sample DB 106 may also be implemented using a storage device (e.g., database server or the like) connected to the analysis apparatus 10 via a communication network, for example.

The analysis apparatus 10 according to this embodiment can implement the various analysis processes described above by having the hardware configuration illustrated in FIG. 4. The hardware configuration illustrated in FIG. 4 is merely an example. The analysis apparatus 10 may have other hardware configurations. For example, the analysis apparatus 10 may include a plurality of processors 205, and may include a plurality of memory devices 206.

The present invention is not limited to the specific disclosure of the embodiment described above and can be modified and changed in various ways, and combined with known techniques, without departing from the scope set forth in the claims.

### REFERENCE SIGNS LIST

10 Analysis apparatus
101 Acquisition unit
102 Conversion unit
103 Computing unit
104 Adjustment unit
105 Effect estimation unit
106 Sample DB
201 Input device
202 Display device
203 External I/F
203a Recording medium
204 Communication I/F
205 Processor
206 Memory device

1. An analysis apparatus for analyzing a causal relationship between an incidence of a predetermined disease and a predetermined intervention, comprising:
a memory; and
a processor configured to execute:
converting a plurality of first parameters indicative of attributes of users belonging to a population, at least two of the parameters having correlations with a predetermined strength, to a plurality of second parameters without the correlations with the predetermined strength with each other;
computing a predetermined score for each of the users, using the plurality of second parameters and a parameter indicative of presence or absence of the intervention; and
clustering the users belonging to the population using the score, to analyze the causal relationship.

2. The analysis apparatus according to claim 1, wherein the converting performs principal component analysis using the plurality of first parameters of the users belonging to the population and converts each one of the plurality of first parameters to a principal component point, to convert the plurality of first parameters to the plurality of second parameters.

3. The analysis apparatus according to claim 1, wherein the clustering clusters the users belonging to the population, using the parameter indicative of presence or absence of the intervention and the score, by matching a set of users with the intervention and a set of users without the intervention based on the score, or by dividing each of the set of users with the intervention and the set of users without the intervention into subclasses based on the score.

4. An analysis apparatus for analyzing a causal relationship between a predetermined event and a predetermined intervention by propensity score analysis, comprising:
a memory; and
a processor configured to execute:
converting a plurality of covariates indicative of attributes of samples belonging to a population, at least two of the covariates having correlations with a predetermined strength, to a plurality of variables without the correlations with the predetermined strength with each other;
computing a propensity score for each of the samples, using the plurality of variables and a treatment variable indicative of presence or absence of the intervention; and
reconstructing a first group and a second group made by classifying the samples based on presence or absence of the intervention using the propensity score so that the covariates become similar.

5. An analysis method executed by an analysis apparatus for analyzing a causal relationship between an incidence of a predetermined disease and a predetermined intervention, the analysis apparatus including a memory and a processor, the analysis method comprising:
converting a plurality of first parameters indicative of attributes of users belonging to a population, at least two of the parameters having correlations with a predetermined strength, to a plurality of second parameters without the correlations with a predetermined strength with each other;
computing a predetermined score for each of the users, using the plurality of second parameters and a parameter indicative of presence or absence of the intervention; and
clustering users belonging to the population using the score, to analyze the causal relationship.

6. A non-transitory computer-readable recording medium having computer-readable instructions stored thereon, which when executed, cause a computer to function as the analysis apparatus according to claim 1.
